(19) United States
(12) Patent Application Publication
(10) Pub. No.: US 2020/0392589 A1
(43) Pub. Date: Dec. 17, 2020

(54) METHODS AND SYSTEMS FOR PROTEOMIC PROFILING AND CHARACTERIZATION

(71) Applicant: Mission Bio, Inc., South San Francisco, CA (US)

(72) Inventors: Dalia Dhingra, San Francisco, CA (US); David Ruff, San Francisco, CA (US); Pedro Mendez, San Mateo, CA (US); Aik Ooi, San Mateo, CA (US)

(21) Appl. No.: 16/839,055

(22) Filed: Apr. 2, 2020

Related U.S. Application Data
(60) Provisional application No. 62/829,291, filed on Apr. 4, 2019.
(60) Provisional application No. 62/828,386, filed on Apr. 2, 2019.
(60) Provisional application No. 62/828,416, filed on Apr. 2, 2019.
(60) Provisional application No. 62/828,420, filed on Apr. 2, 2019.
(60) Provisional application No. 62/829,358, filed on Apr. 4, 2019.

(51) Int. Cl. C12Q 1/6888; C12Q 1/686

(52) U.S. Cl. CPC C12Q 1/6888; C12Q 2565/1015; C12Q 2600/16; C12Q 1/686

(57) ABSTRACT

Provided herein are methods and systems for identifying and characterizing proteins, in particular cell surface proteins, of different cell types at the single-cell level. Also provided are methods and systems for distinguishing cells by their protein expression profiles. Further, methods and systems to quantitate and characterize proteins in single cells at ultrahigh throughput are provided. The methods and systems provided herein are able to sensitively profile all epitopes in a proteome of a single cell.

### RELATED APPLICATIONS

This application takes priority to the following U.S. Provisional Applications U.S. Ser. No. 62/829,291 filed Apr. 4, 2019 and entitled ‘Method, System And Apparatus For Antibody Tag Priming And Genomic Dna Bridge’; U.S. Ser. No. 62/828,386 filed Apr. 2, 2019 and entitled ‘A Complete Solution For Hight Throughput Single Cell Sequencing; U.S. Ser. No. 62/828,416 filed Apr. 2, 2019 and entitled ‘Analytical Methods To Identify Tumor Heterogeneity’; U.S. Ser. No. 62/828,420 filed Apr. 2, 2019 and entitled ‘Method and Apparatus for Universal base library preparation’; and U.S. Ser. No. 62/829,358 filed Apr. 4, 2019 and entitled ‘Method and Apparatus for Fusion in DNA and RNA’, all incorporated by reference herein.

### SEQUENCE LISTING

The instant application contains a Sequence Listing which has been submitted electronically in ASCII format and is hereby incorporated by reference in its entirety. Said ASCII copy, created on Aug. 21, 2020, is named MSB-005WO_SL.txt and is 2,552 bytes in size.

### FIELD

This invention relates generally to the identification, characterization, and profiling of the protein expression pattern or Proteomic analysis of cells, and more particularly to Proteomic analysis in a single cell using unique barcoded nucleotide primers that can be used in an automated system.

### BACKGROUND

Proteins are the primary effectors of cellular function, including cellular metabolism, structural dynamics, and information processing. Proteins are the physical building blocks of cells, comprising the majority of cell mass and carrying out most cell functions, including cell structure dynamics, metabolism, and information processing. They are the molecular machines that convert thermodynamic potential into the energy of living systems. Measuring protein expression and modification is thus important for obtaining an accurate snapshot of cell state and function. A common challenge when measuring proteins at the single-cell level is that most cell systems are heterogeneous, containing massive numbers of molecularly distinct cells. A centimeter-sized tissue volume, for example, can contain billions of cells, each with its own unique spectrum of protein expression and modification; moreover, this underlying cellular heterogeneity can have important consequences on the system as a whole, such as in development, the regulation of the immune system, cancer progression and therapeutic response. For heterogeneous systems like these, methods for high-throughput protein profiling in single cells are necessary.

Profiling proteins in single cells at high throughput requires methods that are sensitive and fast. Flow cytometry with fluorescently-labeled antibodies has been a bedrock in biology for decades because it can sensitively profile proteins in millions of single cells. By labeling antibodies with dyes of different color, profiling can be multiplexed to tens of proteins. By swapping dyes with mass tags and using a mass spectrometer for the readout, multiplexing can be increased to over a hundred antibodies. Nevertheless, while these methods continue to improve in sensitivity and multiplexing, they remain far from enabling the characterization of the entire proteome in single cells, which for humans comprises >20,000 proteins and >100,000 epitopes. A system that could sensitively profile all epitopes in a proteome would be extremely valuable, because it would obviate the need to select which proteins to target. However, existing methods with dye and mass tags are not scalable to the level of full proteome analysis, and in the case of mass-cytometry, destroy the transcriptome during analysis, making it challenging to obtain simultaneous measurements of proteome and transcriptome from the same single cell. (see Shahi, P., Kim, S., Haliburton, J. et al. Abseq: Ultrahigh-throughput single cell protein profiling with droplet microfluidic barcoding. Sci Rep 7, 44447 (2017). https://doi.org/10.1038/srep44447).

Accordingly, the need to characterize proteins, in particular cell surface proteins, of different cell types at the single-cell level is apparent. There is also a need distinguish cells by their protein expression profiles. Additionally, there is a need to detect and quantitate proteins in single cells at ultrahigh throughput. Problematically, the quantitative characterization of proteins at the single-cell level is challenging due to the amount of noise in the readout from signal not attributed to cells. The inventions provided here address these unmet needs.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

In a first aspect, embodiments of the invention are directed to methods of determining and characterizing the protein expression pattern of a single cell.

An exemplary embodiment includes the following: conjugating barcode sequences flanked by PCR priming sites onto antibodies, where a barcode sequence is specific to an antibody; performing a cell identification step using the barcode conjugated antibodies; partitioning or separating individual cells and encapsulating one or more individual cell(s) in a reaction mixture comprising a protease; incubating the encapsulated cell with the protease in the drop to produce a cell lysate; providing one or more nucleic acid amplification primer sets, wherein one or more primer of a primer set comprises a barcode identification sequence associated with an antibody; performing a nucleic acid amplification reaction to produce one or more amplicons; providing an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity and characterizing one or more protein by sequencing a barcode of an amplicon.

Another embodiment comprises a method of determining and characterizing the protein expression pattern of a single cell, the method including independent of order the steps of: conjugating barcode sequences flanked by PCR priming sites onto antibodies, wherein a barcode sequence is specific to an antibody; performing a cell identification step using the barcode conjugated antibodies; partitioning or separating individual cells and encapsulating one or more individual cell(s) in a reaction mixture comprising a protease; incubating the encapsulated cell with the protease in the drop to produce a cell lysate; providing one or more nucleic acid amplification primer sets targeting nucleic acids present in a cell, wherein one or more primer of a primer set includes a barcode identification sequence associated with an antibody; providing one or more nucleic acid amplification primer sets targeting nucleic acids present in a cell, wherein one or more primer of a primer set includes a barcode identification sequence unique to each cell; optionally, performing a reverse transcription polymerase step; performing a nucleic acid amplification reaction to produce one or more amplicons; providing an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity and characterizing one or more protein by sequencing a barcode of an amplicon.

In one exemplary implementation, a reverse primer comprises the following nucleic acid sequence: CTCAACACGGGAAACCTCAC (SEQ ID NO: 1). In one exemplary implementation, a forward primer comprises the following nucleic acid sequence: CGCTCCACCAACTAAGAACG (SEQ ID NO: 2). In one exemplary implementation, a reverse primer comprises the following nucleic acid sequence: TTCCCTCTACACACTGC (SEQ ID NO: 3). In one exemplary implementation, a forward primer comprises the following nucleic acid sequence: ACACCTATTCCAAAATTGACCAC (SEQ ID NO: 4). In one exemplary implementation, a reverse primer comprises the following nucleic acid sequence: CCCGAGTAGCTGGGA TTACA (SEQ ID NO: 5). In one exemplary implementation, a forward primer comprises the following nucleic acid sequence: CCTGAGGTCAGGAGTTC (SEQ ID NO: 6). In one exemplary implementation, a forward barcode primer comprises the following nucleic acid sequence GTACTCGCAGTAGTCCGCTCCACCAACTAAGAACG (SEQ ID NO: 7). In one exemplary implementation, a reverse barcode primer comprises the following nucleic acid sequence:GTCTCGTGGGCTCGGAGATGTGTATAAGAGACAGGGTAAGTGCTGATCTTGGATGT GACG (SEQ ID NO: 8)

Another exemplary embodiment includes adding a barcode identification sequence linked to an antibody, the method includes, independent of order, the steps: performing a barcoding PCR reaction of a target gDNA using a) a primer containing a cell barcode sequence and a PCR handle; b) a primer containing sequence complementary to the target genomic DNA and a PCR handle that is complementary to the primer containing the cell barcode and c) a reverse primer comprising a sequence complementary to the target genomic DNA, an antibody tag sequence, a second PCR handle, and could include a unique molecular tag, to produce an amplicon comprising a cell barcode, a target DNA sequence, an antibody tag with a PCR handle on both the 5′ end and 3′ end; and performing a library creation PCR reaction using a first primers comprising sequencing adapters, sample indexes, and sequences complementary to the two PCR handles produced on the amplicon to produce library comprising sequencing adapters, dual or single sample indexes, a cell barcode, a target DNA sequence, an antibody tag, and could include a unique molecular tag.

Another exemplary embodiment is directed to method for adding a barcode identification sequence linked to an antibody, the method including, independent of order, the following steps: performing a barcoding PCR reaction of a target gDNA using a) a primer containing a cell barcode sequence and a PCR handle; b) a primer containing sequence complementary to the target genomic DNA and a PCR handle that is complementary to the primer containing the cell barcode and c) a reverse primer comprising a sequence complementary to the target genomic DNA, an antibody tag sequence, a second PCR handle, and could include a unique molecular tag, to produce an amplicon comprising a cell barcode, a target DNA sequence, an antibody tag with a PCR handle on both the 5′ end and 3′ end, a first read sequence a first cell barcode, a constant region 1, a second cell bar code, a constant region 2, the forward primer sequence, an insert sequence of length ‘n’, a reverse primer comprising a sequence complementary to the target genomic DNA, a unique molecular identifier, an antibody tag sequence, to a second unique molecular identifier; a second read sequence; and performing a library creation PCR reaction using a first primers comprising sequencing adapters, sample indexes, and sequences complementary to the two PCR handles produced on the amplicon comprising a P5 sequence and a second read sequence and a second primer comprising a second read sequence, and index sequence, and a P7 sequence to produce library comprising sequencing adapters, dual or single sample indexes, a cell barcode, a target DNA sequence, an antibody tag, and could include a unique molecular tag.

In one exemplary implementation, the method further includes preparing an antibody library and a DNA library which can be paired based on the cell barcode.

In one exemplary implementation, the method further includes preparing an antibody library and a RNA library which can be paired based on the cell barcode.

In one exemplary implementation, the method further includes preparing an antibody library, DNA library, and RNA library which can be paired based on the cell barcode.

### BRIEF DESCRIPTION OF THE DRAWINGS

FIG. 1 is a schematic diagram of an approach used in some embodiments. In this Figure, the following designations are used: A-tag=antibody tag; CBC=cell barcode; const1=constant region 1; const2=constant region 2; and Index=sample index.

FIG. 2 shows HS DNA chip data plots from antibody libraries from stained cells, an equal mixture of KG-1 cells and Raji cells. The top panel plot shows results from antibody library 1 (tubes 1-4) using 2 uL of a 530 bp amplicon targeting LINE1,including adaptors (FIG. 2A). The bottom panel plot shows results from antibody library 2 (tubes 5-8) using 2 uL of a 530 bp amplicon targeting LINE1, including adaptors (FIG. 2B).

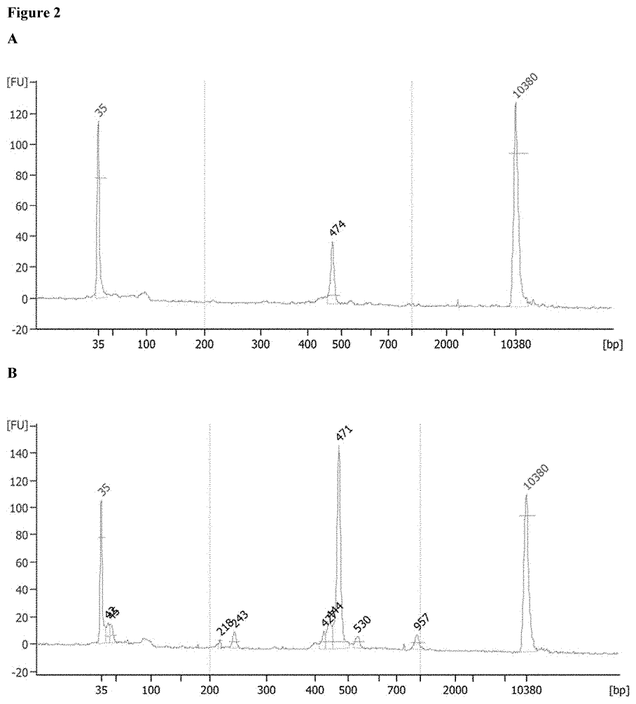

FIG. 3 shows HS DNA chip data plots from the corresponding DNA libraries from stained cells, an equal mixture of KG-1 cells and Raji cells. The top panel plot shows results from DNA library 1 (tubes 1-4) using 2 uL of a 50 amplicon panel targeting mutations common in acute myeloid leukemia. The bottom panel plot shows results from DNA library 2 (tubes 5-8) using 2 uL of a 50 amplicon panel targeting mutations common in acute myeloid leukemia.

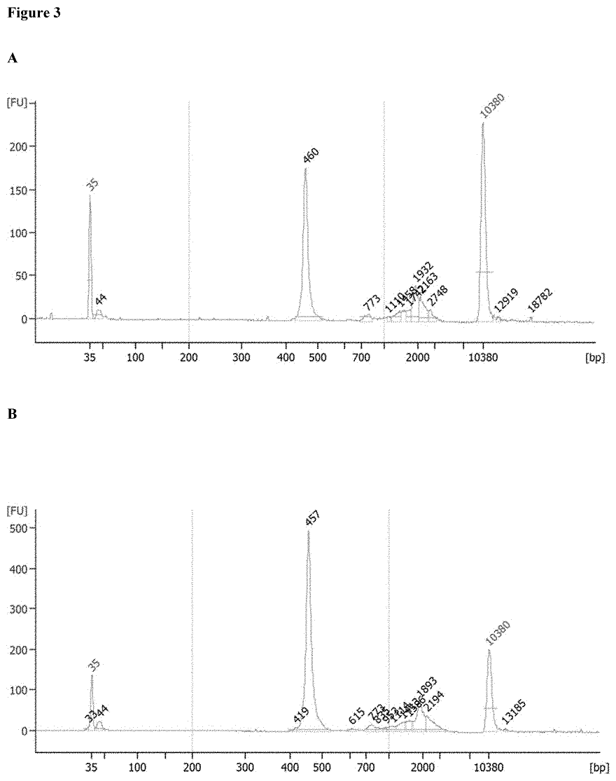

FIG. 4 is a graph showing the alignment of the amplicon to LINE1 after the cell barcode and antibody tag are trimmed. 99.4% of the reads with both a cell barcode antibody tag aligns to LINE1.

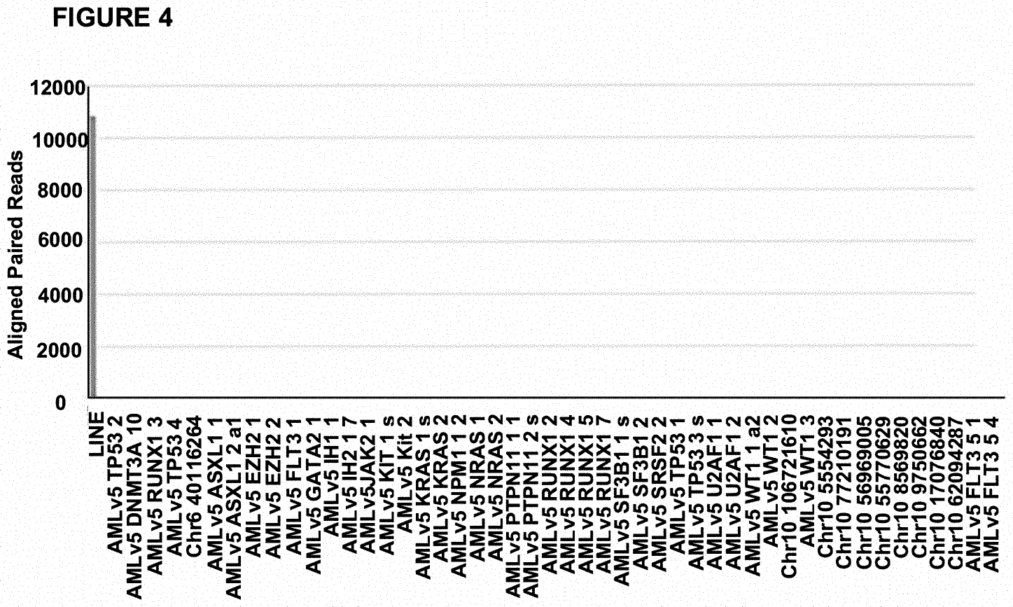

FIG. 5 is a table of data showing the distribution of amplicons across hg19. 1098 paired reads from the antibody library were aligned to hg19. Reads aligned to every chromosome with varying lengths.

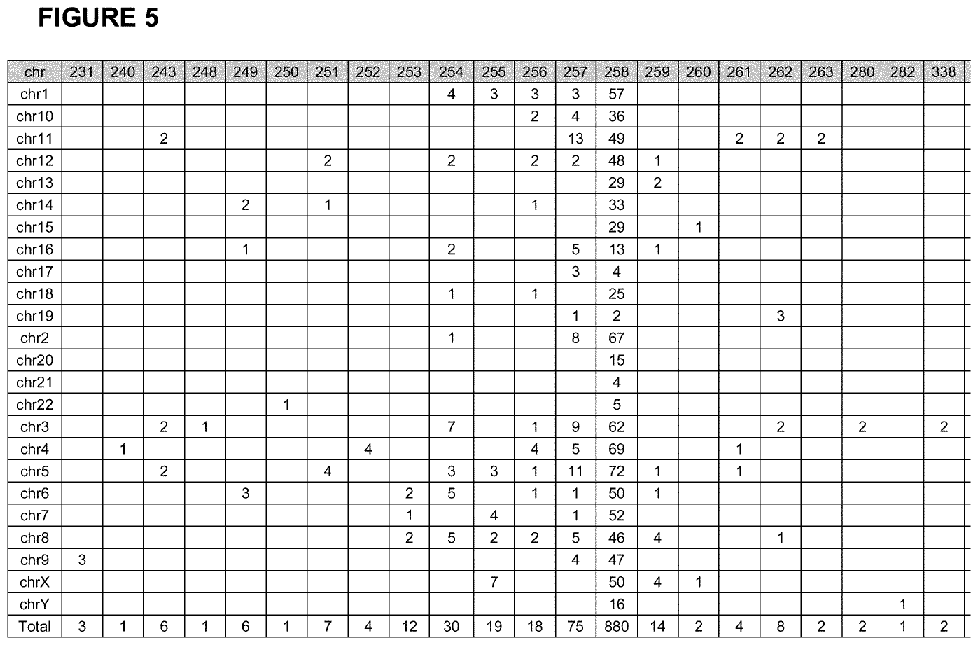

FIG. 6 is a table where a subsample of paired reads from an antibody library were used to analyze the antibody calls. Based on the tag sequence, a cell was either positive for CD34, CD19, or both. The cells input to the reaction were KG-1 and Raji mixed in an equal ratio where KG-1 cells are positive for CD34 and Raji cells are positive for CD 19. The majority of antibodies were unique between cell barcodes as should be observed with sequenced single cell antibody libraries from stained cells.

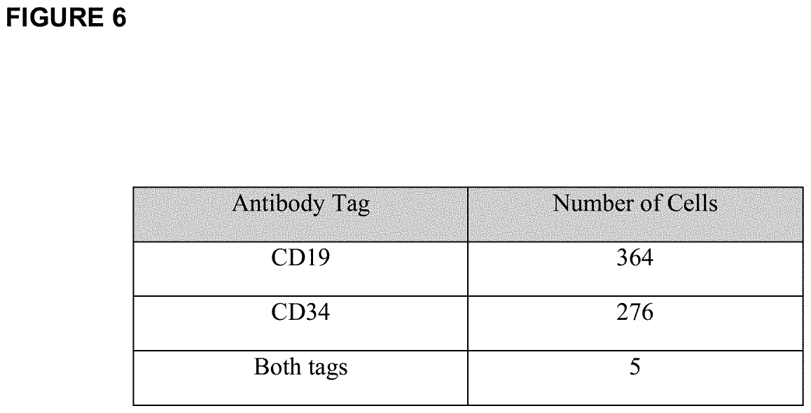

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

A ‘barcode’ nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

A barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri™ beads to tag and then later identify each droplet's amplicon content. The use of barcodes is described in U.S. patent application Ser. No. 15/940,850 filed Mar. 29, 2018 by Abate, A. et al., entitled ‘Sequencing of Nucleic Acids via Barcoding in Discrete Entities’, incorporated by reference herein.

In some embodiments, it may be advantageous to introduce barcodes into discrete entities, e.g., microdroplets, on the surface of a bead, such as a solid polymer bead or a hydrogel bead. These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may be utilized, such as a PCR-based assay, e.g., quantitative PCR (qPCR), or an isothermal amplification may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

The number of amplification/PCR primers that may be added to a microdroplet may vary. The number of amplification or PCR primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

One or both primers of a primer set may comprise a barcode sequence. In some embodiments, one or both primers comprise a barcode sequence and a unique molecular identifier (UMI). In some embodiments, where both a UMI and a nucleic acid barcode sequence are utilized, the UMI is incorporated into the target nucleic acid or an amplification product thereof prior to the incorporation of the nucleic acid barcode sequence. In some embodiments, where both a UMI and a nucleic acid barcode sequence are utilized, the nucleic acid barcode sequence is incorporated into the UMI or an amplification product thereof subsequent to the incorporation of the UMI into a target nucleic acid or an amplification product thereof

One or both primer of a primer set may also be attached or conjugated to an affinity reagent. In some embodiments, individual cells, for example, are isolated in discrete entities, e.g., droplets. These cells may be lysed and their nucleic acids barcoded. This process can be performed on a large number of single cells in discrete entities with unique barcode sequences enabling subsequent deconvolution of mixed sequence reads by barcode to obtain single cell information. This approach provides a way to group together nucleic acids originating from large numbers of single cells. Additionally, affinity reagents such as antibodies can be conjugated with nucleic acid labels, e.g., oligonucleotides including barcodes, which can be used to identify antibody type, e.g., the target specificity of an antibody. These reagents can then be used to bind to the proteins within or on cells, thereby associating the nucleic acids carried by the affinity reagents to the cells to which they are bound. These cells can then be processed through a barcoding workflow as described herein to attach barcodes to the nucleic acid labels on the affinity reagents. Techniques of library preparation, sequencing, and bioinformatics may then be used to group the sequences according to cell/discrete entity barcodes. Any suitable affinity reagent that can bind to or recognize a biological sample or portion or component thereof, such as a protein, a molecule, or complexes thereof, may be utilized in connection with these methods. The affinity reagents may be labeled with nucleic acid sequences that relates their identity, e.g., the target specificity of the antibodies, permitting their detection and quantitation using the barcoding and sequencing methods described herein. Exemplary affinity reagents can include, for example, antibodies, antibody fragments, Fabs, scFvs, peptides, drugs, etc. or combinations thereof. The affinity reagents, e.g., antibodies, can be expressed by one or more organisms or provided using a biological synthesis technique, such as phage, mRNA, or ribosome display. The affinity reagents may also be generated via chemical or biochemical means, such as by chemical linkage using N-Hydroxysuccinimide (NETS), click chemistry, or streptavidin-biotin interaction, for example. The oligo-affinity reagent conjugates can also be generated by attaching oligos to affinity reagents and hybridizing, ligating, and/or extending via polymerase, etc., additional oligos to the previously conjugated oligos. An advantage of affinity reagent labeling with nucleic acids is that it permits highly multiplexed analysis of biological samples. For example, large mixtures of antibodies or binding reagents recognizing a variety of targets in a sample can be mixed together, each labeled with its own nucleic acid sequence. This cocktail can then be reacted to the sample and subjected to a barcoding workflow as described herein to recover information about which reagents bound, their quantity, and how this varies among the different entities in the sample, such as among single cells. The above approach can be applied to a variety of molecular targets, including samples including one or more of cells, peptides, proteins, macromolecules, macromolecular complexes, etc. The sample can be subjected to conventional processing for analysis, such as fixation and permeabilization, aiding binding of the affinity reagents. To obtain highly accurate quantitation, the unique molecular identifier (UMI) techniques described herein can also be used so that affinity reagent molecules are counted accurately. This can be accomplished in a number of ways, including by synthesizing UMIs onto the labels attached to each affinity reagent before, during, or after conjugation, or by attaching the UMIs microfluidically when the reagents are used. Similar methods of generating the barcodes, for example, using combinatorial barcode techniques as applied to single cell sequencing and described herein, are applicable to the affinity reagent technique. These techniques enable the analysis of proteins and/or epitopes in a variety of biological samples to perform, for example, mapping of epitopes or post translational modifications in proteins and other entities or performing single cell proteomics. For example, using the methods described herein, it is possible to generate a library of labeled affinity reagents that detect an epitope in all proteins in the proteome of an organism, label those epitopes with the reagents, and apply the barcoding and sequencing techniques described herein to detect and accurately quantitate the labels associated with these epitopes.

Primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. Primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

In some implementations, solid supports, beads, and the like are coated with affinity reagents. Affinity reagents include, without limitation, antigens, antibodies or aptamers with specific binding affinity for a target molecule. The affinity reagents bind to one or more targets within the single cell entities. Affinity reagents are often detectably labeled (e.g., with a fluorophore) Affinity reagents are sometimes labeled with unique barcodes, oligonucleotide sequences, or UMI's.

In some implementations, a RT/PCR polymerase reaction and amplification is performed, for example in the reaction mixture, an addition to the reaction mixture, or added to a portion of the reaction mixture.

In one particular implementation, a solid support contains a plurality of affinity reagents, each specific for a different target molecule. Affinity reagents that bind a specific target molecule are collectively labeled with the same oligonucleotide sequence such that affinity molecules with different binding affinities for different targets are labeled with different oligonucleotide sequences. In this way, target molecules within a single target entity are differentially labeled in these implements.

### Antibody TAGs, Genomic DNA Bridges, and Proteomics

A first objective of some embodiments herein is to provide a sensitive, accurate, and comprehensive characterization of proteins in large numbers of single cells.

Certain methods provided herein utilize specific antibodies to detect epitopes of interest. In some embodiments, antibodies are labeled with sequence tags that can be read out with microfluidic barcoding and DNA sequencing. This and related implementations are used herein to characterize cell surface proteins of different cell types at the single-cell level.

In some embodiments, a barcode identity is encoded by its full nucleobase sequence and thus confers a combinatorial tag space far exceeding what is possible with conventional approaches using fluorescence. A modest tag length often bases provides over a million unique sequences, sufficient to label an antibody against every epitope in the human proteome. Indeed, with this approach, the limit to multiplexing is not the availability of unique tag sequences but, rather, that of specific antibodies that can detect the epitopes of interest in a multiplexed reaction.

In some implementations, cells are bound with antibodies against the different target epitopes, as in conventional immunostaining, except that the antibodies are labeled with barcodes.

In practice, when an antibody binds its target the antibody barcode tag is carried with it and thus allows the presence of the antibody and the presence of a cell to be inferred. In some implementations, counting antibody barcode tags provides an estimate of the different epitopes present in the cell.

Other embodiments implementations are used to distinguish particular cells by their protein expression profiles. Some embodiments of DNA-tagged antibodies provided herein have multiple advantages for profiling proteins in single cells.

A primary advantage of these implementations is the ability to amplify low-abundance tags to make them detectable with sequencing. Another advantage in some implementations is the capability of using molecular indices for quantitative results. Some implementations also have essentially limitless multiplexing capabilities.

Some embodiments utilize solid beads having an alternate chemistry where the primers to be used are in solution and contain a PCR annealing sequence embedded, or ‘handle’, that allows hybridization to primers. In some implementations, the handle is a specific tail 5′ upstream of the target sequence and this handle is complimentary to bead barcoded oligo and serves as a PCR extension bridge to link the target amplicon to the bead barcode library primer sequence. The solid beads may contain primers that can anneal to the PCR handle on the primers.

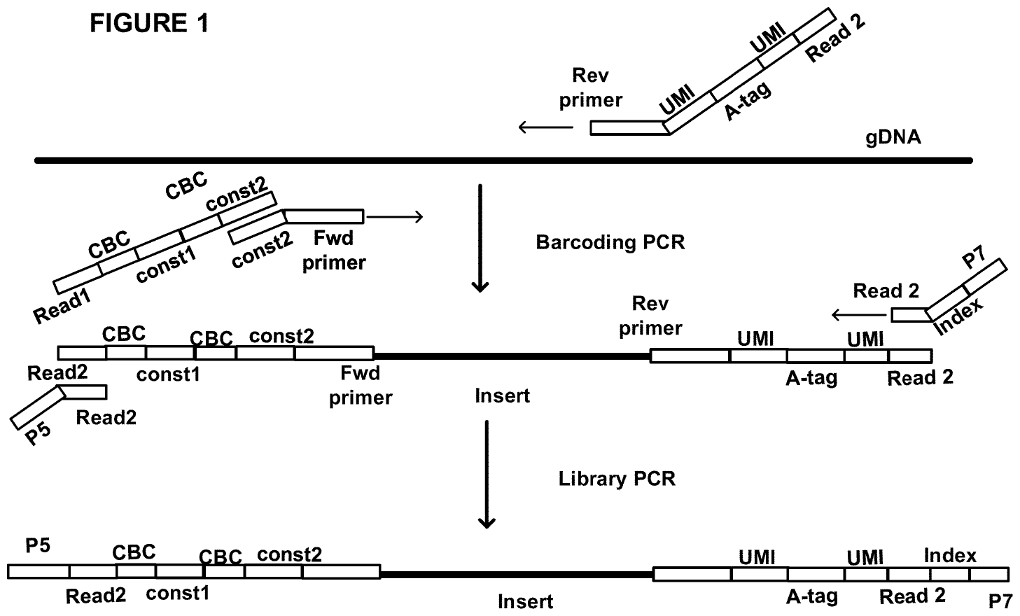

One particular embodiment is for a method for adding a barcode identification sequence linked to an antibody, the method comprising the steps: i) an initial hybridization of a target gDNA to a) a forward primer comprising a first read sequence adjacent to cell barcode and a handle sequence b) a reverse primer comprising a sequence complementary to the target genomic DNA, which could include a unique molecular tag; which is adjacent to a second handle sequence, and performing a PCR reaction. The resulting amplicon comprises a PCR handle sequence adjacent to cell barcode sequences, which is attached to the forward primer sequence, which is adjacent to an insert sequence of length ‘n’, which is adjacent to a reverse primer comprising a sequence complementary to the target genomic DNA, optionally, unique molecular tags, antibody tag sequences, and a second PCR handle. An additional library creation PCR step is typically used in some embodiments to further attach indexing and identification sequences (see for example FIG. 1).

Antibody libraries can be created from antibody stained cells, and these can identify and characterized by sequencing.

In another aspect, some implementations provided herein can be used to detect and characterize the DNA and protein expression pattern in single cell.

In another aspect, some implementations provided herein can be used to detect and characterize the RNA and protein expression pattern in single cell.

In another aspect, some implementations provided herein can be used to detect and characterize the DNA, RNA, and protein expression pattern in single cell.

In some implementations, the target nucleic acid sequence can be used based on length and sequence to identify unique antibody tags.

In some implementations, certain affinity reagent barcoding techniques described herein can be used to detect and quantitate protein-protein interactions. For example, proteins that interact can be labeled with nucleic acid sequences and reacted with one another. If the proteins interact by, for example, binding one another, their associated labels are localized to the bound complex, whereas proteins that do not interact will remain unbound from one another.

The sample can then be isolated in discrete entities, such as microfluidic droplets, and subjected to fusion amplification/PCR or barcoding of the nucleic acid labels. In the case that proteins interact, a given barcode group will contain nucleic acids including the labels of both interacting proteins, since those nucleic acids would have ended up in the same compartment and been barcoded by the same barcode sequence. In contrast, proteins that do not interact will statistically end up in different compartments and, thus, will not cluster into the same barcode group post sequencing. This allows identification of which proteins interact by clustering the data according to barcode and detecting all affinity reagent labels in the group.

Certain embodiments the invention provide methods for linking and amplifying nucleic acids conjugated to proteins, such as antibodies, enzymes, receptors, and the like. An exemplary method includes: (a) incubating a population of nucleic acid barcode sequence-conjugated proteins under conditions sufficient for a plurality of the proteins to interact, bringing the nucleic acid barcode sequences on the interacting proteins in proximity to each other; (b) encapsulating the population of nucleic acid barcode sequence-conjugated proteins in a plurality of discrete entities such that interacting proteins are co-encapsulated, if present; (c) using a microfluidic device to combine in a discrete entity contents of one of the plurality of first discrete entities and reagents sufficient for amplification and linkage of the nucleic acid barcode sequences on the interacting proteins, if present; and (d) subjecting the discrete entity to conditions sufficient for the amplification and linkage of the nucleic acid barcode sequences on the interacting proteins, if present.

Some embodiments utilize solid beads having an alternate chemistry where the primers to be used are in solution and contain a PCR annealing sequence embedded, or ‘handle’, that allows hybridization to primers. In some implementations, the handle is a specific tail 5′ upstream of the target sequence and this handle is complimentary to bead barcoded oligo and serves as a PCR extension bridge to link the target amplicon to the bead barcode library primer sequence. The solid beads may contain primers that can anneal to the PCR handle on the primers.

Other aspects of the invention may be described in the follow embodiments:
1. An apparatus or system for performing a method described herein.
2. A composition or reaction mixture for performing a method described herein.
3. An antibody library generated by methods described herein.
4. A genomic library generated by methods described herein.
5. A transcriptome library generated according to a method described herein.
6. An antibody library, genomic, and transcriptome library generated according to a method described herein.
7. A kit for performing a method described herein.
8. A cell population selected by the methods described herein.
9. A system for molecular profiling for performing a method herein.
10. A method for preparing an antibody library and a DNA library which can be paired based on the cell barcode.
11. A method for preparing an antibody library and a RNA library which can be paired based on the cell barcode.
12. A method for preparing an antibody library, DNA library, and RNA library which can be paired based on the cell barcode.

The following Examples are included for illustration and not limitation.

### Example I

### Antibody TAG Priming and Genomic DNA Bridge

The disclosed embodiments generally relate to using an antibody tag as a primer during single cell polymerase chain reaction (PCR) resulting in amplicons being generated only in the presence of a cell. Among others, the disclosed embodiments provide an alternative approach to Proteomic analysis which can be used to minimize background noise.

In some implementations, analysis and characterization of a cellular proteome is performed by initially conjugating antibody tags flanked by PCR priming sites onto antibodies. The antibody tags are composed of a DNA sequence specific to that antibody. These conjugated antibodies are used to stain cells, which are then run through the Tapestri™ platform. As a cell is partitioned into droplets, its corresponding antibodies are as well. During the barcoding PCR where the gDNA or RNA targets are amplified, the antibody tags are also amplified. These amplicons are then made into libraries for sequencing. In droplets containing a bead but no cell, any antibody that has dissociated from the cells can still be amplified and is assigned a cell barcode. If a fraction of the sequencing run is taken up by the background noise then these reads must be filtered out of the dataset during analysis.

In this Example, we use we use DNA from the cell as our targeted nucleic acid and the oligo on the antibody is the primer. This approach uniquely eliminates the amplification of antibody tags from antibodies that have dissociated from cells and thus maximizes sequencing read efficiency.

In an exemplary method according to one embodiment of the disclosure, the antibodies can be conjugated with antibody tags flanked by a PCR handle and a reverse gene specific primer (5′-PCR handle rev—antibody tag—gene specific reverse primer-3′). The antibody tags will still be composed of a DNA sequence specific to that antibody. In certain embodiments, the corresponding gene specific forward primer (5′-PCR handle fwd—gene specific forward primer-3′) is included in the forward primer mix used in barcoding PCR. This forward mix can be attached to the bead or present in solution. The PCR handle for the forward primer can be altered depending on the chemistry used (see FIG. 1).

After cell staining and lysing, during the barcoding PCR, only if nucleic acid (gDNA or RNA) is present would the antibody tag primer hybridize and extend. This extension can be performed by a DNA polymerase or a reverse transcriptase. The corresponding forward primer will prime on the DNA copy or cDNA then extend through the antibody tag sequence. This cycle results in an amplicon containing the antibody tag with the PCR handles needed for library preparation. If gDNA or RNA is not present in the droplet, the antibody tag primer would not extend. As a result, only droplets containing a bead and a cell would produce library from the antibody tags.

With this approach, read 1 can sequence through the cell barcode, forward primer and amplicon while read 2 can sequence the antibody tag, reverse primer, and amplicon. Only droplets with cells present will produce amplicon with cell barcode and antibody tag that can be amplified further in library PCR. This will minimize the noise from droplets that do not contain cells.

Assuming ˜100 copies of an antibody tag attached to a single cell (diploid genome) within a Tapestri™ emulsion (˜350 pL), the concentration of primer and template are within the range used for multiplexed PCR.

In one embodiment, gene specific priming sites for the antibody tags can be selected based on copies and prevalence of the antibodies. For instance, a single copy gene target may be selected for a highly prevalent antibody. For other antibodies, targets with multiple copies may be selected to increase priming sites, such as 18s, LINE1, or ALU. Since the copies of these targets are known, they can be used to normalize the resulting sequencing data. In the case of 18s, due to the high degree of homology within eukaryotes, an antibody tag primer universal for human, mice, and rat was designed.

The gene specific priming sites may also be designed for the antibody tags so the amplicon will contain variable regions of the genome. As the amplicon is sequenced, the variable region can be used as a molecular tag to distinguish PCR copies. For example, with ALU115 primers and LINE1 primers, there are ˜100,000 copies and ˜7000 copies per haploid respectively. With the antibodies priming at these various sites, the amplicons produced can have variable sequences. These variable sequences can be collapsed for each antibody tag to produce unique antibody reads.

In an implementation, the following gene specific primers are used.

18s—400 copies per haploid genome

reverse:
(SEQ ID NO: 1)
CTCAACACGGGAAACCTCAC
forward:
(SEQ ID NO: 2)
CGCTCCACCAACTAAGAACG

LINE1—˜7000 copies per haploid genome

reverse:
(SEQ ID NO: 3)
TTCCCTCTACACACTGC
forward:
(SEQ ID NO: 4)
ACACCTATTCCAAAATTGACCAC

ALU115—˜100,000 copies per haploid genome

reverse:
(SEQ ID NO: 5)
CCCGAGTAGCTGGGATTACA
forward:
(SEQ ID NO: 6)
CCTGAGGTCAGGAGTTC

### Barcoding Primers:

Reverse primer: 5-PCR handle rev—antibody tag—gene specific reverse primer-3′

Forward primer: 5-PCR handle fwd—gene specific forward primer-3′

### Example 18s Barcoding Primers:

Reverse primer:
(SEQ ID NO: 8)
GTCTCGTGGGCTCGGAGATGTGTATAAGAGACAGGG
TAAGTGCTGATCTTGGATGTGACG
(SEQ ID NO: 9)
TCTCAACACGGGAAACCTCAC
Forward primer:
(SEQ ID NO: 7)
GTACTCGCAGTAGTCCGCTCCACCAACTAAGAACG

### Sequencing Reads:

Read 1=cell barcode+PCR handle+gene specific forward primer+−insert

Read 2=antibody tag+gene specific reverse primer+insert

TABLE 1
Sequencing Results

| | Total | R1 line1 | R1 line1 | Paired line1 | Paired line1 | Paired line1 |
|---|---|---|---|---|---|---|
| Library | Reads | reads | reads (%) | reads | (%) | aligned to hg19 |
| Al | 24267 | 1106 | 4.56% | 1101 | 3.8% | 99.9% |

Table 1 shows that the single cell library produced using antibody tags as priming sites for LINE1 in the human genome produced reads whose two sequencing reads could be paired and aligned to the expected target sequence. These aligned libraries had the expected structure.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of determining and characterizing the protein expression pattern of a single cell, the method comprising the steps of:
a) conjugating barcode sequences flanked by PCR priming sites onto antibodies, wherein a barcode sequence is specific to an antibody;
b) performing a cell identification step using the barcode conjugated antibodies;
c) partitioning or separating individual cells and encapsulating one or more individual cell(s) in a reaction mixture comprising a protease;
d) incubating the encapsulated cell with the protease in the drop to produce a cell lysate;
e) providing one or more nucleic acid amplification primer sets targeting nucleic acids present in a cell, wherein one or more primer of a primer set includes a barcode identification sequence associated with an antibody;
f) providing one or more nucleic acid amplification primer sets targeting nucleic acids present in a cell, wherein one or more primer of a primer set includes a barcode identification sequence unique to each cell;
g) performing a nucleic acid amplification reaction to produce one or more amplicons;
h) providing an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence;
i) contacting an affinity reagent to the amplification product comprising amplicons of one or more target nucleic acid sequence under conditions sufficient for binding of the affinity reagent to the target nucleic acid to form an affinity reagent bound target nucleic acid; and
j) determining the identity and characterizing one or more protein by sequencing a barcode of an amplicon.

2. A method of claim 1, wherein a reverse primer comprises the following nucleic acid sequence: CTCAACACGGGAAACCTCAC (SEQ ID NO: 1)

3. A method of claim 1, wherein a forward primer comprises the following nucleic acid sequence: CGCTCCACCAACTAAGAACG (SEQ ID NO: 2).

4. A method of claim 1, wherein a reverse primer comprises the following nucleic acid sequence: TTCCCTCTACACACTGC (SEQ ID NO: 3).

5. A method of claim 1, wherein a forward primer comprises the following nucleic acid sequence: ACACCTATTCCAAAATTGACCAC (SEQ ID NO: 4).

6. A method of claim 1, wherein a reverse primer comprises the following nucleic acid sequence: CCCGAGTAGCTGGGA TTACA (SEQ ID NO: 5).

7. A method of claim 1, wherein a forward primer comprises the following nucleic acid sequence: CCTGAGGTCAGGAGTTC (SEQ ID NO: 6).

8. A method of claim 1, wherein a forward barcode primer comprises the following nucleic acid sequence GTACTCGCAGTAGTCCGCTCCACCAACTAAGAACG (SEQ ID NO: 7)

9. A method of claim 1, wherein a reverse barcode primer comprises the following nucleic acid sequence: GTCTCGTGGGCTCGGAGATGTGTATAAGAGACAGGGTAAGTGCTGATCTTG GATGTGACG (SEQ ID NO: 8)

10. A method for adding a barcode identification sequence linked to an antibody, the method comprising the steps:
i) performing a barcoding PCR reaction of a target gDNA using a) a primer containing a cell barcode sequence and a PCR handle; b) a primer containing sequence complementary to the target genomic DNA and a PCR handle that is complementary to the primer containing the cell barcode and c) a reverse primer comprising a sequence complementary to the target genomic DNA, an antibody tag sequence, a second PCR handle, and could include a unique molecular tag, to produce an amplicon comprising a cell barcode, a target DNA sequence, an antibody tag with a PCR handle on both the 5′ end and 3′ end; and
ii) performing a library creation PCR reaction using a first primers comprising sequencing adapters, sample indexes, and sequences complementary to the two PCR handles produced on the amplicon to produce library comprising sequencing adapters, dual or single sample indexes, a cell barcode, a target DNA sequence, an antibody tag, and could include a unique molecular tag.

11. A method for adding a barcode identification sequence linked to an antibody, the method comprising the steps:
i) performing a barcoding PCR reaction of a target gDNA using a) a primer containing a cell barcode sequence and a PCR handle; b) a primer containing sequence complementary to the target genomic DNA and a PCR handle that is complementary to the primer containing the cell barcode and c) a reverse primer comprising a sequence complementary to the target genomic DNA, an antibody tag sequence, a second PCR handle, and could include a unique molecular tag, to produce an amplicon comprising a cell barcode, a target DNA sequence, an antibody tag with a PCR handle on both the 5′ end and 3′ end, a first read sequence a first cell barcode, a constant region 1, a second cell bar code, a constant region 2, the forward primer sequence, an insert sequence of length ‘n’, a reverse primer comprising a sequence complementary to the target genomic DNA, a unique molecular identifier, an antibody tag sequence, to a second unique molecular identifier; a second read sequence; and
ii) performing a library creation PCR reaction using a first primers comprising sequencing adapters, sample indexes, and sequences complementary to the two PCR handles produced on the amplicon comprising a P5 sequence and a second read sequence and a second primer comprising a second read sequence, and index sequence, and a P7 sequence to produce library comprising sequencing adapters, dual or single sample indexes, a cell barcode, a target DNA sequence, an antibody tag, and could include a unique molecular tag.

12. A method according to claim 1, comprising performing reverse transcription to produce a reverse transcription product.

13. A method according to claim 1, comprising performing reverse transcription to produce a reverse transcription product before a nucleic acid amplification step.

14. A method according to claim 1, comprising performing reverse transcription on the RNA to produce a reverse transcription product and amplifying the reverse transcription product, wherein performing reverse transcription and amplifying occur in a single step.

15. A method according to claim 1, further comprising performing a nucleic acid sequencing reaction of an amplification product.

16. A method according to claim 1, wherein the affinity reagent comprises a bead or the like.

17. A method according to claim 1, comprising determining and characterizing the expression of one or more cell surface protein.

18. A method according to claim 1 further comprising preparing an antibody library and a DNA library which can be paired based on the cell barcode.

19. A method according to claim 1 further comprising preparing an antibody library and a RNA library which can be paired based on the cell barcode.

20. A method according to claim 1 further comprising preparing an antibody library, DNA library, and RNA library which can be paired based on the cell barcode.


SEQUENCE LISTING (9 sequences)

SEQ ID NO: 1 - DNA, 20 bp, Artificial Sequence
  Description of Artificial Sequence Synthetic oligonucleotide
ctcaacacgggaaacctcac

SEQ ID NO: 2 - DNA, 20 bp, Artificial Sequence
  Description of Artificial Sequence Synthetic oligonucleotide
cgctccaccaactaagaacg

SEQ ID NO: 3 - DNA, 17 bp, Artificial Sequence
  Description of Artificial Sequence Synthetic oligonucleotide
ttccctctacacactgc

SEQ ID NO: 4 - DNA, 23 bp, Artificial Sequence
  Description of Artificial Sequence Synthetic oligonucleotide
acacctattccaaaattgaccac

SEQ ID NO: 5 - DNA, 20 bp, Artificial Sequence
  Description of Artificial Sequence Synthetic oligonucleotide
cccgagtagctgggattaca

SEQ ID NO: 6 - DNA, 17 bp, Artificial Sequence
  Description of Artificial Sequence Synthetic oligonucleotide
cctgaggtcaggagttc

SEQ ID NO: 7 - DNA, 35 bp, Artificial Sequence
  Description of Artificial Sequence Synthetic oligonucleotide
gtactcgcagtagtccgctccaccaactaagaacg

SEQ ID NO: 8 - DNA, 60 bp, Artificial Sequence
  Description of Artificial Sequence Synthetic oligonucleotide
gtctcgtgggctcggagatgtgtataagagacagggtaagtgctgatcttggatgtgacg

SEQ ID NO: 9 - DNA, 21 bp, Artificial Sequence
  Description of Artificial Sequence Synthetic oligonucleotide
tctcaacacgggaaacctcac